(19) United States
(12) Patent Application Publication
(10) Pub. No.: US 2021/0007696 A1
(43) Pub. Date: Jan. 14, 2021

(54) MOTION DETECTION AND CORRECTION IN MRI AND OTHER IMAGING APPLICATIONS USING MEMS SENSORS

(71) Applicant: Resonance Technology, Inc., Northridge, CA (US)

(72) Inventors: Mokhtar Ziarati, North Hollywood, CA (US); Parisa Ziarati, Granada Hills, CA (US)

(21) Appl. No.: 16/874,066

(22) Filed: May 14, 2020

Related U.S. Application Data
(60) Provisional application No. 62/872,649, filed on Jul. 10, 2019.

(51) Int. Cl. A61B 6/00; A61B 5/055; A61B 5/00; A61B 6/03; G01R 33/28; G01R 33/565; G01P 13/00

(52) U.S. Cl. CPC A61B 6/527; A61B 5/055; A61B 5/721; A61B 6/032; A61B 6/037; A61B 5/6803; G01R 33/4806; G01R 33/283; A61B 5/0042; G01R 33/56509; G01P 13/00; A61B 2562/028; A61B 2562/0219; A61B 5/682

(57) ABSTRACT

A motion sensor for a patient in an imaging application. A MEMS motion sensor is arranged for mounting or attachment to the patient's head, the motion sensor configured to detect the patient's motion and provide sensor signals to a utilization device such as a motion compensation processor to compensate patient images for the detected motion or a controller to send a message to the patient when motion is detected.

### BACKGROUND

Patient motion in imaging modalities such as CT, PET and MRI has presented great challenges. Due to nature of the imaging data acquisition process, a significant amount of time can elapse between different samples. This leaves many image sequences vulnerable to patient motion. The resulting increases in misregistration between images can greatly impair the diagnostic quality of an MRI examination. The medical condition of a patient, such as tremor, pain, or mental status, often prevents even willing patients from holding still. Despite the fact that there have been numerous patents and publications addressing the problem, there is no practical and cost-effective method in the market, to be compatible with clinical applications.

A popular method that is being used is an optical motion correction approach. In this method usually one or 3 cameras are set up either outside of the imaging machine bore and with the aid of mirrors. The camera(s) is focused on a retro-grade reflector or marker which is attached to the subject's forehead or other body part for each camera. The camera observes the marker and extracts its pose. The pose from the camera is sent to the scanner control and processing computer, allowing for correction of scan planes and position for motion of the patient.

There are disadvantages to the camera approach to detect movement of the patient in the MRI bore. The MRI head coil is not open to the side. Attachment of other apparatus to it may de-tune the coil and cause image artifacts. The attachments to the head may be bulky and difficult or impossible to use in the clinical application. This system set up is very expensive, and the set up could take hours.

### BRIEF DESCRIPTION OF THE DRAWINGS

Features and advantages of the disclosure will readily be appreciated by persons skilled in the art from the following detailed description when read in conjunction with the drawing wherein:

FIG. 1A-1D diagrammatically illustrate a motion sensor in accordance with the invention on a patient's head within a head coil of an MRI system.

FIG. 2 is a diagrammatic illustration of the sensor device of FIGS. 1A-1D shown on the patient's head, and schematically showing other elements of the sensor system and the motion compensation system.

FIGS. 3A-3D illustrate an alternate embodiment of a motion sensor device mounted on a bite bar for use by a patient in an MRI application.

FIGS. 4A and 4B illustrate respective front and back views of another embodiment of a motion sensor system integrated with a set of video goggles configured to be worn by a patient in an MRI or other imaging procedure. FIG. 4C is a schematic block diagram of a system using the goggles in an MRI imaging application.

FIG. 4D is a simplified flow diagram illustrating an example of use of the googles to monitor patient movement and encourage stillness.

### DETAILED DESCRIPTION

In the following detailed description and in the several figures of the drawing, like elements are identified with like reference numerals. The figures may not be to scale, and relative feature sizes may be exaggerated for illustrative purposes.

Embodiments of the system provide advantages in sensing movement of the patient's head in imaging applications, including increased accuracy, cost effectiveness and ease of use. Embodiments of the system require very little set up in order to use it for clinical and fMRI applications. Embodiments of the invention are not limited to MRI applications, and may be used for other imaging modalities such as CT, PET, SPECT scanner and digital angiography.

By using Micro Electro-Mechanical System (MEMS) technology in accordance with an aspect of the invention, the cameras and bulky assemblies used in known systems can be replaced with a system utilizing one or more very small MEMS sensors, typically not bigger than a few mm by a few mm.

A MEMS sensor is a chip-based technology, wherein a mass is suspended between a pair of capacitive plates. When tilt is applied to the sensor, the suspended mass creates a difference in electrical potential, measured as a change in capacitance. The signal of the MEMS sensor may be amplified to create a stable output signal, e.g. in digital, 4-20 mA or VDC. In a general sense, embodiments of a sensor system include a MEMS sensor with a power source (if needed), a device for mounting, attaching or supporting the sensor relative to the patient's head and a communication link for conveying the sensor signals to a motion compensation processor or other utilization system.

Motion correction per se is well known in the imaging art. Examples of systems employing motion correction are described in “Motion Correction in MRI of the brain,” F Godenschweger et al 2016 Phys. Med. Biol. 61 R32, Number 5; and “An embedded optical tracking system for motion-corrected magnetic resonance imaging at 7T,” J. Schulz et al., MAGMA Magnetic Resonance Materials in Physics Biology and Medicine (2012), 25:443-453.

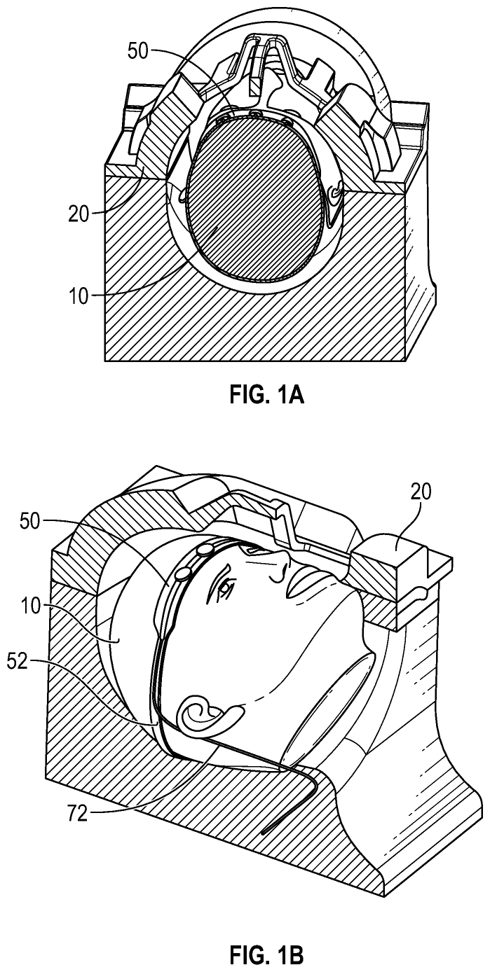

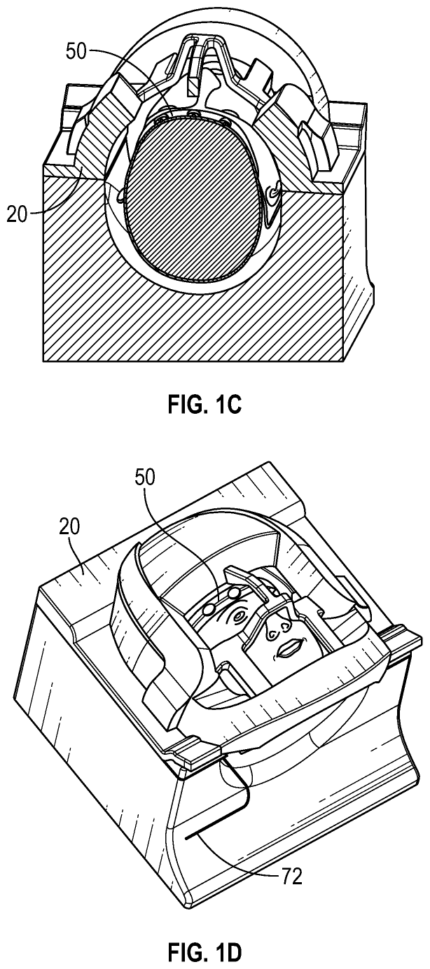

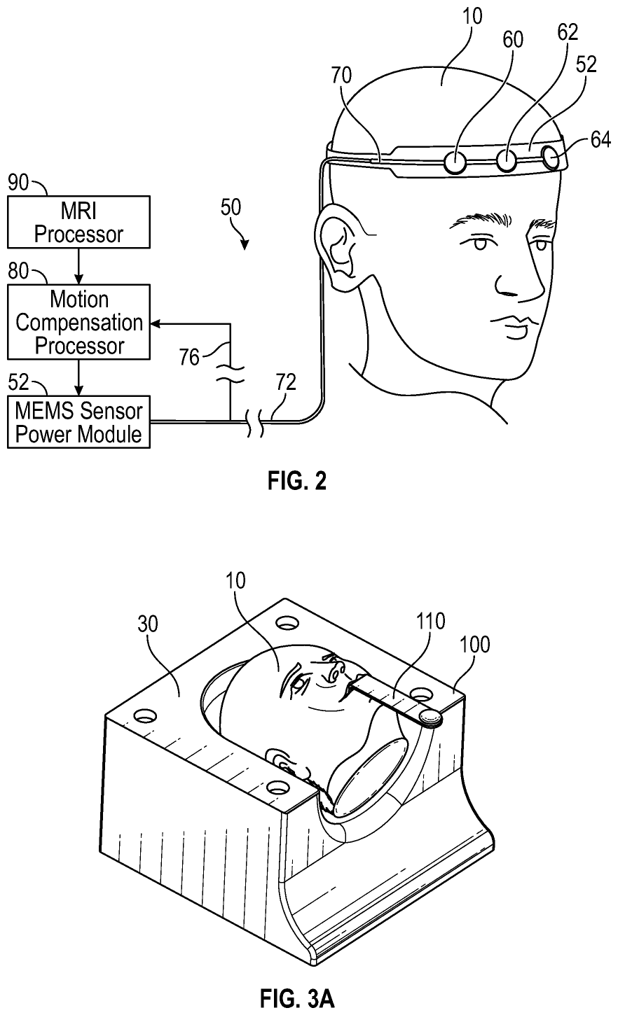

An exemplary embodiment of the sensor system and its placement on the patient's body is illustrated in FIGS. 1A-1D and 2. The sensor system 50 in this embodiment includes a head band 52 as the attaching device and three MEMS sensor devices 60, 62 and 64 mounted in spaced relation on the head band 52. The sensor devices are electrically connected to non-ferrous wiring 70 which includes a wiring portion 72 extending from the head band for connection to a communication link 76 for conveying the sensor signals to a motion compensation controller 80. The controller 80 in turn is connected to the MRI processor 90 and provides motion signals to the MRI processor indicative of patient motion in real time. The wiring 72 is also connected to the MEMS sensor power module 52 for supplying electrical power to the MEMS sensors 60, 62, 64. Exemplary MEMS sensors typically use 5 VDC as a bias voltage. The power module is typically located well away from the MRI tube so as not to interfere with the imaging process.

In this embodiment, the MEMS devices 60, 62, 64 may be commercially available devices, such as the SCC1300-D04 gyro sensor and accelerometer system by Murate Electronics, and the LSM6DSO system, an always-on 3D accelerometer and 3D gyroscope module, by STI. In an exemplary embodiment, the sensor output signals are in digital form, and are representative of changes in position with respect to pre-set references in any or all three (X, Y, Z) directions. For improved accuracy, three sensors 60, 62, 64 may be used, each dedicated to changes in X, Y or Z direction. At least one sensor is used that provides data representative of changes in the X, Y and Z to measure the motion so that imaging corrections to compensate the motion may be determined. While any MEMS sensor may be used, 3-axis sensors are preferred.

The sensor 50 in this embodiment is connected through wiring 72 to a power module 52, which typically will be positioned either in the MRI magnet room well away from the MRI bore, or even in the MRI control room. The motion signals from the sensor 50 are connected through a communication link 76 to a motion compensation controller 80, which processes the sensor signals to determine appropriate motion compensation signals to the MRI processor 90 to compensate the MRI images for the sensed motion of the patent 10 inside the MRI tube or head coil. Both the compensation controller 80 and the MRI processor 90 will typically be located outside the MRI magnet room, typically in the MRI control room.

FIGS. 1A-1D illustrate the positioning of the patient 10 wearing the sensor 50 for a brain scan in an MRI head coil 20. Not shown in these figures is the MRI bore.

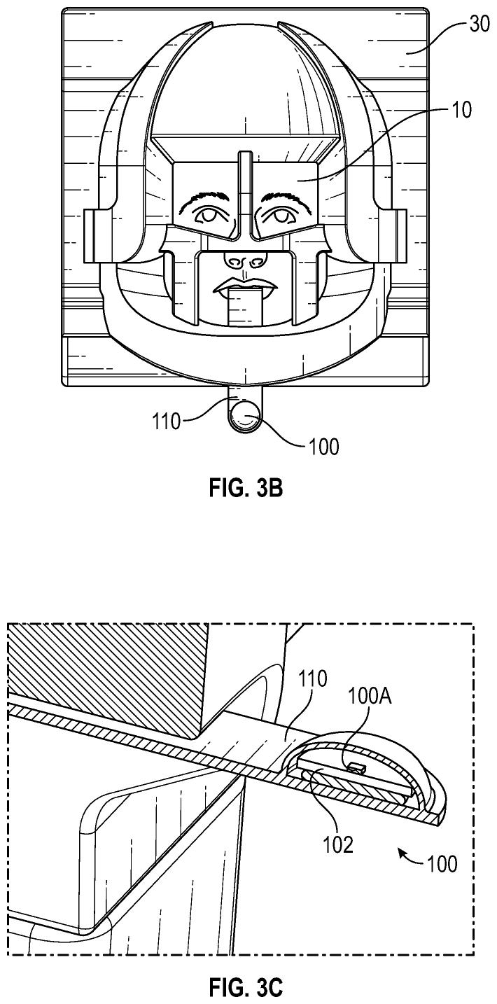

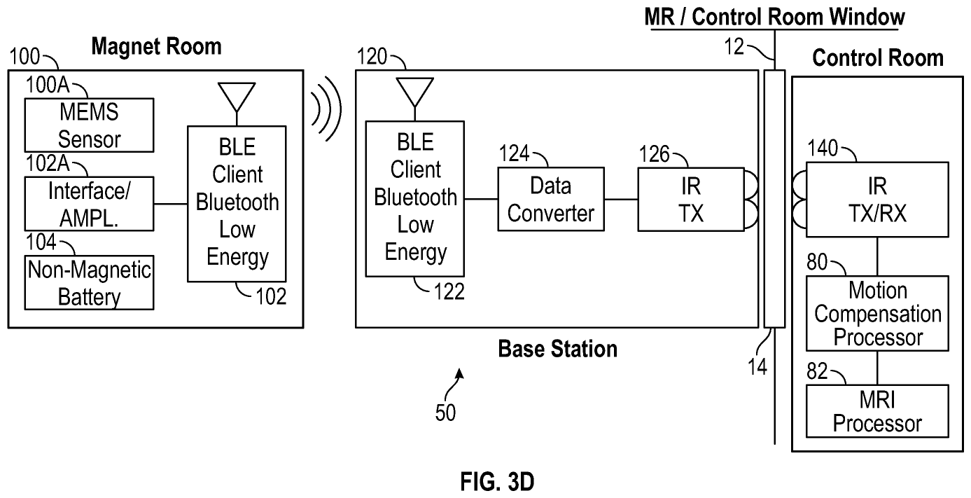

An alternate embodiment of a motion sensor module 100 is illustrated in FIGS. 3A-3D. The sensor module 100 is placed at the end of a bite bar 110. In this embodiment, the sensor module includes MEMS sensor 100A, a single 3 axis MEMS sensor which is used to measure the movement of the patient in all three axes. The bite bar 110 is disposable in this exemplary embodiment and the sensor module 100 is removably attached to the tip of the bite bar. This embodiment employs a Blue Tooth (TM) module 102 and a non-magnetic battery 104 to provide power to the module 100 to wirelessly transmit the recorded data to a receiver or base station in the magnet room and at same time transmit it with an IR transmitter through the control room window 14 to the MRI motion processor 80 and MRI processor 82 as a real time feedback for image correction. U.S. Ser. No. 10/083,598 describes a system which utilizes Blue Tooth™ devices and IR transmission to provide a communication link from a device in the magnet room through the magnet room window to a receiver in the control room. The entire contents of U.S. Pat. No. 100,835,398 are incorporated herein by this reference. The bite bar 110 is fabricated of a non-magnetic material.

As shown in FIGS. 3A and 3B, the patent 10 bites down on one end of the bite bar 110, which has a 90-degree bend so that the main portion of the bite bar does not protrude upwardly in the MRI tube. In the simplified FIGS. 3A and 3B, the patient's head is positioned with the head coil 30 in place.

FIG. 3D illustrates an exemplary operating environment for the MEMS sensor system in general, and the embodiment of FIGS. 3A-3C in particular. An MRI installation includes a magnet room in which the MRI magnet is disposed. The room walls, floor and ceiling are typically shielded to prevent passage of electromagnetic signals or energy. A control room is separated from the magnet room by a wall 12, in which a window 14 is installed. Typically, the window is shielded to prevent RF energy to pass through it, while allowing light energy, including IR, to pass through. The motion compensation processor 80 and MRI processor 82 are disposed in the control room. In some embodiments, the functions of the processor 80 may be incorporated into the MRI processor 82.

Still referring to FIG. 3D, the sensor module 100 includes the MEMS sensor 100A, the BLE client Bluetooth™ and the non-magnetic battery 104. The BLE client receives the MEMS sensor signals from sensor interface/amplifier 102A, and wirelessly transmits the sensor signals to the base station, which is situated near the window 14. The base station receives the transmitted signals from sensor 100 in the magnet room, converts it, and transfers it via IR through the glass of the MR/Control room window 14 to the processors 80, 82 located in the control room. Thus, the base station, in this example, includes a BLE Server 122, which receives and transfers the sensor signals to a data converter 124, which converts the sensor signals and prepares them for transmission via IR to the control room. The base station IR transmitter 126 transmits and receives serial messages to the control room. The emitter of the device 126 can be placed adjacent the window as generally indicated in FIG. 3D. The base station may be powered by a non-magnetic battery for convenience, and will have a non-magnetic housing, e.g. plastic or aluminum. The base station is typically placed at a sufficient distance from the magnet that it will not have any significant impact on the MRI imaging.

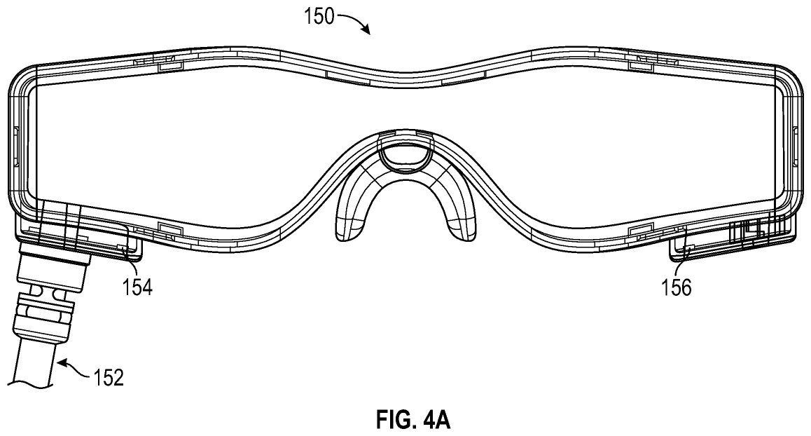

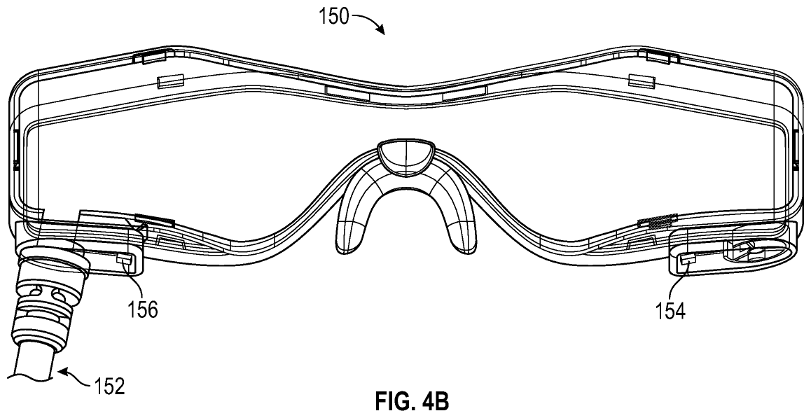

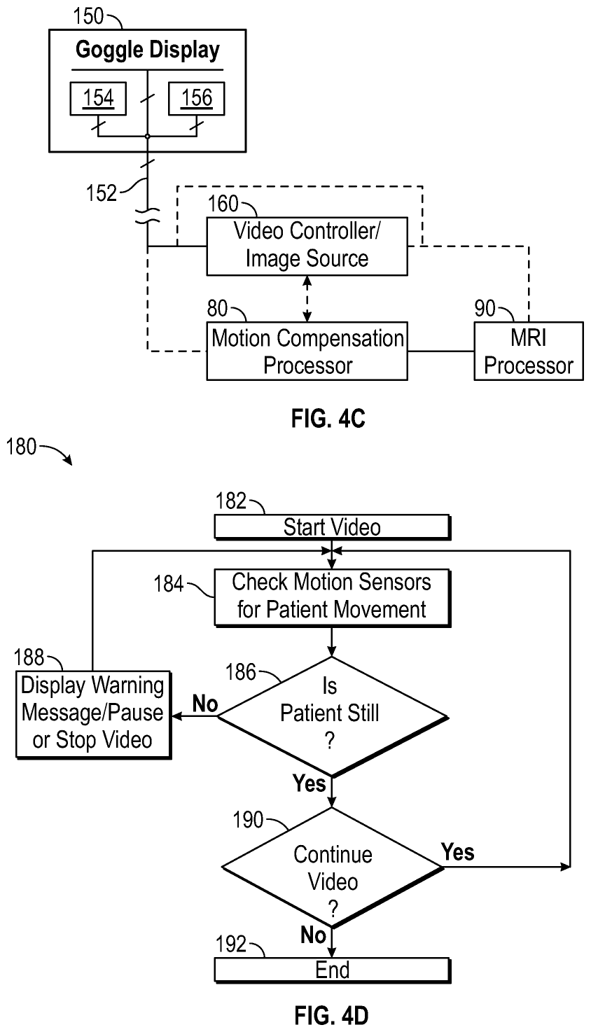

FIGS. 4A-4D illustrate a further embodiment of a motion-detecting sensor for use in an imaging application. As is known, video goggles are used to show images and video to patients undergoing MRI procedures. Video display devices for use in MRI applications are described, for example, in U.S. Pat. Nos. 9,787,750 and 9,454,008. The purpose of the video goggles is for the patient to watch movies or images to reduce the claustrophobic effect, or, in cases of fMRI applications (brain research), the scientist can present various paradigms to the subject patient for brain mapping. The goggles are non-magnetic and designed for use in MRI and other imaging equipment. In accordance with an aspect of the invention, the MEMS sensor(s) may be integrated with the goggle to measure the patient motion and/or to give feedback to the patient not to move during the procedure, e.g. by showing messages to the patient through the goggles if the patient moves his or her head or simply turn off or pause the video if the patient moves. This will encourage patients, particularly children, to stay still during the image scan. In the latter instance, the patient would have to remain still to watch the video.

Still referring to FIGS. 4A-4D, a set of video goggles 150 is illustrated. The goggles in this embodiment are connected by a non-magnetic cable to a video controller/image source 160 (FIG. 4C). In other embodiments, the signal connection may be wireless, e.g. through a Bluetooth™ connection. Left and right MEMS sensors 154, 156 are mounted to the goggle frames adjacent the respective left and right eyepieces of the goggles, and electrically connected through the cable 152 to the motion compensation processor or to the video controller controller/image source, depending on the application. The entire goggle, its housing and the electronics are non-magnetic.

FIG. 4C diagrammatically illustrates the goggle 150 in communication with the video controller/image source 160. Typically the video controller/image source 160 will be in the MRI control room, with communication link 152 carrying the video/image signals to the goggles 150, and carrying the motion signals from the sensors 154, 156 to the controller/image source 160 and optionally to the motion compensation processor 80 as in the case with the embodiments of FIGS. 1A-3D. In the case in which motion compensation is not applied, the motion sensor signals may be employed merely to control the image source, to encourage the patient to remain still. In that case, the sensor signals may be applied directly to the MRI processor 90 so that the MRI processor may pause image collection or processing while the patient is not still.

FIG. 4D illustrates an exemplary process flow 150 for the goggle embodiment. At 182, a video is started with the patient in the MRI tube and wearing the goggles. The start may be initiated by the patient, or by the MRI technologist. At 184, the motion sensor signals are checked for patient movement. If the patient is still at 186, and if the video should continue (e.g. if the image scan is still underway), the video continues, and operation returns to 184. If not, operation ends at 192. If at 186 the patient is not still, the video controller/image source 160 generates a warning message on the goggles for the patient to remain still. The video may also be paused or ended. Operation returns to 184, for the check of the motion sensors. The process illustrated is merely exemplary.

While the motion sensor embodiments described with respect to FIGS. 1A-2 and FIGS. 4A-4D have been described as using wired communication links, each may alternatively be used with wireless communication links, e.g. as described with respect to the embodiment of FIGS. 3A-3D.

Although the foregoing has been a description and illustration of specific embodiments of the subject matter, various modifications and changes thereto can be made by persons skilled in the art without departing from the scope and spirit of the invention.

What is claimed is:

1. A motion sensor for a patient in an imaging application utilizing a patient imaging system, comprising:
a MEMS motion sensor system arranged for mounting or attachment to the patient's head;
a device for mounting or attaching or securing the MEMS sensor to the patient's head during an imaging procedure;
wherein the MEMS motion sensor system is configured to detect motion of the patient's motion during the imaging procedure and to provide sensor signals to compensate patient images for the detected motion.

2. The motion sensor of claim 1, wherein the imaging application is one of MRI, fMRI, CT, PET.

3. The motion sensor of claim 1, wherein the attachment device comprises a head band structure configured to be worn by the patient, the MEMS sensor attached to the headband structure.

4. The motion sensor of claim 1, wherein the MEMS sensor system includes a plurality of MEMS sensors.

5. The motion sensor of claim 1, wherein the MEMS sensor system is configured to detect motion in each of the X, Y and Z axis.

6. The motion sensor of claim 1, wherein the attachment device comprises a bite bar having one end configured for being held in the patient's mouth, the MEMS sensor system attached to the bite bar.

7. The motion sensor of claim 1, wherein the MEMS sensor system is integrated with a video goggle configured to be worn by the patient in the imaging application, the attachment device comprising the video goggle.

8. The motion sensor of claim 1, further comprising a communication link configured to communicate the sensor signals to a motion compensation processor.

9. The motions sensor of claim 8, wherein the communication link includes a non-ferrous wiring connection.

10. The motion sensor of claim 8, wherein the communication link includes a wireless signal communication link for transmitting the sensor signals to a base station in communication with the motion compensation processor.

11. A motion sensor system for a patient in an imaging application utilizing a patient imaging system, comprising:
a motion sensor arranged for mounting or attachment to the patient's head, the sensor comprising one or more MEMS gyro sensor and accelerometer modules;
a device for mounting, attaching or securing the motion sensor in relation to the patient's head during an imaging procedure;
wherein the motion sensor is configured to detect motion of the patient's motion during the imaging procedure and to provide sensor signals indicative of the patient's motion;
a communication link for delivering the sensor signals to a motion compensation processor of the imaging application, to compensate patient images for the detected motion.

12. The system of claim 11, wherein the communication link includes a non-ferrous wiring connection to the motion sensor.

13. The system of claim 11, wherein the communication link includes a wireless signal communication link configured to transmit wireless signals representative of the sensor signals to a base station in communication with the motion compensation processor.

14. The system of claim 11, wherein the attachment device comprises a head band structure configured to be worn by the patient, the MEMS sensor attached to the headband structure.

15. The system of claim 11, wherein the MEMS sensor includes a plurality of MEMS sensors.

16. The system of claim 11, wherein the one or more MEMS modules is configured to detect motion in each of the X, Y and Z axis.

17. The system of claim 11, wherein the attachment device comprises a non-magnetic bite bar having one end configured for being held in the patient's mouth, the MEMS sensor system attached to the bite bar.

18. The system of claim 11, wherein the MEMS sensor is integrated with a video goggle configured to be worn by the patient in the imaging application, the attachment device comprising the video goggle.

19. The system of claim 11, wherein the imaging application is one of MRI, fMRI, CT and PET.

20. A motion sensor system for a patient in an imaging application utilizing a patient imaging system, comprising:
a motion sensor arranged for mounting or attachment to the patient's head, the sensor comprising one or more MEMS gyro sensor and accelerometer modules, wherein the MEMS sensor is integrated with a video goggle configured to be worn by the patient in the imaging application, the goggle configured to deliver video images to the patient undergoing imaging;
wherein the motion sensor is configured to detect motion of the patient's head during the imaging procedure and to provide sensor signals indicative of the patient's motion;
a communication link for delivering the sensor signals to a utilization device, including one or more of a video controller/image source and a motion compensation processor of the imaging application, to compensate patient images for the detected motion.

21. The system of claim 20, wherein the utilization device includes the video controller/image source, and is responsive to motion of the patient's head during an imaging procedure to send a warning message to the goggle to display a warning message to the patent and/or to pause or stop the video being presented to the patient.
